(19) United States
(12) Patent Application Publication
(10) Pub. No.: US 2021/0371888 A1
(43) Pub. Date: Dec. 2, 2021

(54) CONTINUOUS BIODIESEL SYNTHESIS PROCESS BY TRANSESTERIFICATION WITH SUPERCRITICAL METHANOL

(71) Applicant: GROSS DEL SURESTE S.A. DE C.V., Campeche (MX)

(72) Inventor: Antonio José De Jesús De San Juan Bosco ECHEVARRIA PARRÉS, Campeche (MX)

(21) Appl. No.: 16/487,839

(22) PCT Filed: Feb. 12, 2018
(86) PCT No.: PCT/MX2018/050005

§ 371 Date: Jul. 16, 2021

(30) Foreign Application Priority Data
Feb. 22, 2017 (MX) MX/A/2017/002340

(51) Int. Cl. C12P 7/64; C12M 1/107; B01D 3/14; B01D 5/00; C12M 1/00; C12M 1/34

(52) U.S. Cl. CPC C12P 7/649; C12M 21/04; B01D 3/14; B01D 5/0015; C12R 2001/89; C12M 43/02; C12M 41/12; C12M 41/40; C12M 29/14

(57) ABSTRACT

A continuous process for the synthesis of biodiesel from microalgae oil by transesterification with supercritical methanol, where the synthesis of biodiesel is carried out in a single transesterification step in a reactor that operates continuously, said process being ideal to be carried out at industrial scale since it can provide a continuous flow of biodiesel.

### BACKGROUND OF THE INVENTION

### A. Field of the Invention

The present invention is related to biodiesel synthesis processes from organic matter by means of supercritical fluids, and more particularly, to a continuous process for the synthesis of biodiesel from microalgae oil by transesterification with supercritical methanol.

### B. Description of Related Art

In the art, there are known a great variety of processes to produce biodiesel from organic matter such as, for example, seaweed.

Conventional processes for the production of biodiesel comprise batch processes by transesterification of vegetable fats/oils that use an alkaline catalyst, followed by steps destined to remove the catalyst and saponified products of the free fatty acids.

In addition, in these processes an additional amount of catalyst is required to neutralize the free fatty acids of an oil having a high content of free fatty acids, which lengthens the process and reduces the yield of the process, since no methyl esters are produced from fatty acids.

To solve these problems, processes have been developed for the production of biodiesel by transesterification with supercritical methanol without using catalysts. Examples of these processes are described in the following documents: “Non-catalytic biofuel production with super critical methanol technologies”, Shiro Saka, Dadan Kusdiana and Eiji Minami, Department of Socio-Environmental Energy Sciences, Graduate School of Energy Sciences, Kyoto University, Japan, Journal of Scientific and Industrial Research, vol. 65, May 2006, pp. 420-425; and “Effects of water on the production of biodiesel fuel by treatment with supercritical methanol”, Dadan Kusdiana, Shiro Saka, Bioresource Technology 91 (2004), pp 289-295.

Both documents describe processes for the production of biodiesel using supercritical methanol without using catalysts, wherein triglycerides present in oils/fats are converted to fatty acid methyl esters without any type of catalysts. Specifically, Shiro's paper describes a first one-step process and a second two-step process.

The one-step process described in the Shiro document, comprises carrying out transesterification of oils/fats with supercritical methanol (350° C./20M Pa) in a batch reactor at a temperature of between 300° C. to 350° C. wherein the triglycerides in the oils/fats are converted into methyl esters of fatty oils (biodiesel) due to their methanolysis ability, producing glycerin as a by-product.

The two-step process described in the Shiro document comprises firstly hydrolyzing the oils/fats in subcritical water in a batch reactor at a temperature between 270° C. to 300° C. and subsequently the esterification of the hydrolyzed oils/fats. with supercritical methanol (270° C./10 MPa) in a reactor at a temperature of 270° C. and a pressure of between 7-15 MPa wherein the triglycerides in the oils/fats are converted into methyl esters of fatty oils (biodiesel), producing glycerin as a by-product.

The two-step process has the advantage that glycerin is separated in the first hydrolysis step, which makes it possible for the synthesis of biodiesel to be carried out by means of esterification under more moderate reaction conditions, although the fact of having the hydrolysis step prior to the synthesis of biodiesel by esterification, makes it more expensive to operate due to the extra equipment and energy required for the synthesis of biodiesel

Therefore, it is more desirable to operate the simplified one-step process wherein the synthesis is carried out by transesterification, however, because the process is designed to be batch operated using a batch reactor, the yield that provide such a process is insufficient to bring it to commercial scale.

It is therefore desirable to have a biodiesel synthesis process that does not need to use catalysts and that can operate continuously in such a way that it can provide a continuous flow of biodiesel as a product.

In view of the aforementioned needs, the applicant developed a continuous process for the synthesis of biodiesel from microalgae oil by transesterification with supercritical methanol. In the process developed by the applicant, the synthesis of biodiesel is carried out in a single transesterification step in a reactor that operates continuously, said process being ideal to be carried out in an industrial scale since it can provide a continuous flow of biodiesel.

Additionally, the process of the present invention includes subsequent steps for the removal of glycerin on a continuous basis.

### SUMMARY OF THE INVENTION

It is therefore a main object of the present invention, to provide a continuous process for the synthesis of biodiesel from microalgae oil by transesterification with supercritical methanol.

It is another main object of the present invention, to provide a continuous process for the synthesis of biodiesel of the nature described above, wherein the synthesis of biodiesel is carried out in a single transesterification step in a reactor that operates continuously, said process being ideal to be carried out in an industrial way since it can provide a continuous flow of biodiesel.

These and other objects and advantages of the present invention will become apparent to those of ordinary skill in the art from the following detailed description of the invention.

### BRIEF DESCRIPTION OF THE DRAWINGS

FIG. 1 is a diagram of the process of the present invention.

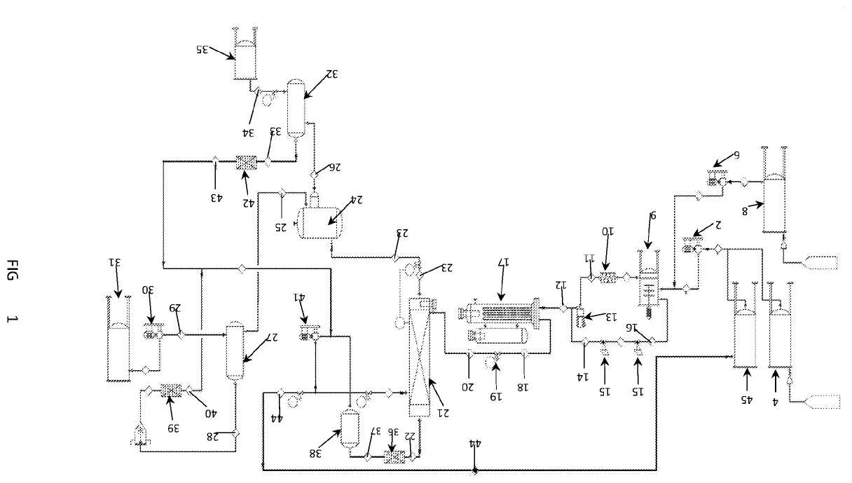

FIG. 2 is a perspective view of the continuous flow reactor of the method of the present invention showing its internal components.

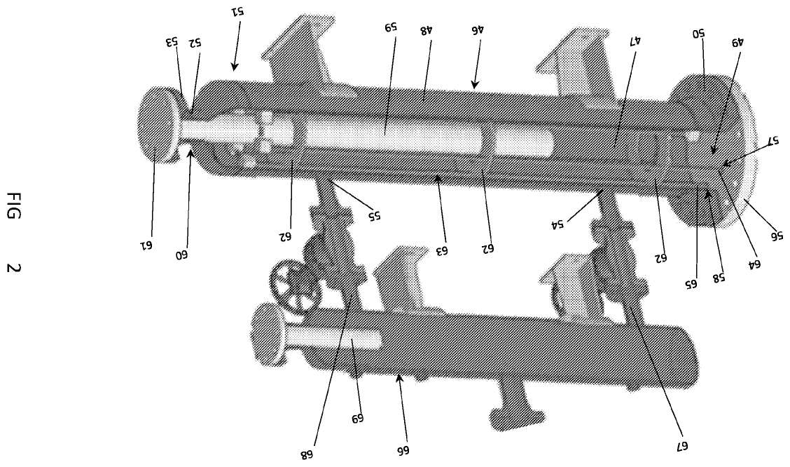

FIG. 3 is a front view of the inner tubular core of the reactor of the present invention.

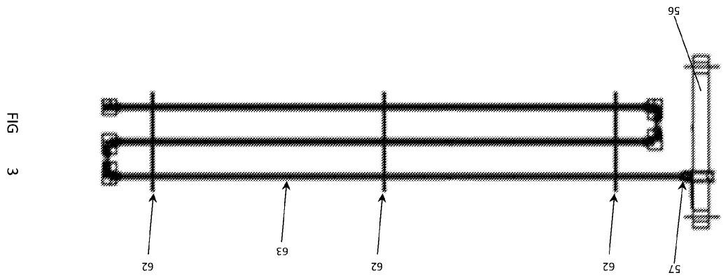

### DETAILED DESCRIPTION OF THE INVENTION

The continuous process for the synthesis of biodiesel from microalgae by transesterification with supercritical methanol will now be described making reference first to a general embodiment thereof and later to specific embodiments thereof.

In a general embodiment, the continuous process for the synthesis of biodiesel from microalgae by transesterification with supercritical methanol of the present invention comprises the steps of:

a) mixing and homogenization a stream of microalgae oil and methanol in a ratio of 1 to 32 with respect to the mass flow (although other proportions may be possible within a deviation of +−15), at room temperature to create a mixing stream homogeneous oil of microalgae and methanol by: sending the mixture to a stirring tank; and its subsequent sending to a static mixer to obtain a stream of homogeneous mixture of microalgae oil and ethanol;

b) continuously processing the stream of homogeneous mixture of microalgae oil and methanol by transesterification within a continuous flow reactor at a pressure of between 370 kg/cm²g to 380 kg/cm²g (preferably 370 kg/cm²g) and at a temperature between 350° C. and 380° C. preferably 350° C., where the stream of the homogeneous mixture enters the reactor at a temperature of approximately 58.02° C. (which is reached by induction), a pressure of between 370 kg/cm²g to 380 kg/cm²g (preferably 370 kg/cm²g) and with a mass flow of 33 kg/h (which may vary depending on the reactor) in order to obtain a mixture of biodiesel, methanol and glycerin that leaves the reactor at a pressure between 370 kg/cm²g to 380 kg/cm²g (preferably 370 kg/cm²g), a temperature of 362.60° C. and with a mass flow of 33 kg/h, which can vary depending on the reactor;

c) depressurizing the mixture of biodiesel, methanol and glycerin that leaves the reactor to a pressure of 2 kg/cm²g and lower the temperature to 131° C.;

d) separating the methanol from the mixture obtained in step c) by feeding it to a distillation column, where the methanol-rich vapor phase comprising the overhead stream is separated at the top, and the reaction products at the bottom in liquid phase comprising biodiesel and glycerin which form the bottom stream, and wherein the overhead stream of the column is cooled to a temperature of 73.05° C.;

e) separating the crude biodiesel in the light liquid phase, glycerin in the heavy liquid phase and traces of methanol in the vapor phase from the mixture of biodiesel and glycerin that comprises the bottom stream obtained in step c), by feeding them to a three-phase separator that works at a temperature of 135° C. and a pressure of 0.50 kg/cm²g;

f) purifying the crude biodiesel in light liquid phase obtained in step e) that contains traces of methanol by feeding it to a two-phase separator operating at a temperature of 44.47° C. where the methanol is separated and purified biodiesel is obtained, which is the final product of the process.

Additionally, in said general embodiment, the process of the present invention comprises the following secondary stages:
condensing the methanol-rich vapors obtained in step d) by feeding them into a plate exchanger for subsequent storage and disposal;
purifying the glycerin obtained in step e) that contains traces of methanol in a separator that works at 44.47° C. and at a pressure of 0.50 kg/cm²g where the traces of methanol are separated from the glycerin to obtain pure glycerin for later storage and disposal;
condensing the methanol-rich vapors obtained in the purification of glycerin by feeding it into a plate exchanger for subsequent storage and disposal.

In a specific embodiment, the continuous process for the synthesis of biodiesel of the present invention starts from microalgae oil which, preferably but not limited to, has the following components:

| Component | Mass Fraction |
|---|---|
| Oleic acid | 0.025 |
| Palmitic triglyceride | 0.95 |
| Palmitic acid | 0.025 |

In such specific embodiment the continuous process for the synthesis of biodiesel of the present invention comprises the steps of:

a) creating a stream of methanol (1) at a pressure of 0.5 kg/cm²g by means of a centrifugal pump (2) that draws methanol (3) from a storage tank (4); and

creating a stream of microalgae oil (triglyceride) (5) at a pressure of 0.5 kg/cm²g by means of a centrifugal pump (6) that draws microalgae oil (7) from a storage tank (8), wherein the proportion of methanol to microalgae oil is from 1 to 32 with respect to the mass flow of both streams and wherein the conditions of both streams (1), (5) are as follows:

| Condition | Microalgae oil | Methanol |
|---|---|---|
| Pressure, kg/cm²g | 0.00 | 0.00 |
| Temperature, ° C. | Ambient | Ambient |
| Mass flow, kg/h | 1.00 | 32.00 |

b) mixing and homogenizing the methanol and microalgae oil streams by continuously feeding the methanol (1) and algae oil (5) streams continuously to a stirring tank (9) and to an adjoining static mixer (10) to produce a homogenized mixture of methanol and microalgae oil (11), where the residence time of methane and microalgae oil in the stirring tank and static mixer is approximately 15 minutes. It is necessary to note that, in this preferred embodiment, said time is the time it takes to empty the tank under the indicated operating conditions, but the residence time can be from one minute;

c) creating a pressurized stream of the homogenized mixture of methanol and microalgae oil (12) obtained in step b) by extracting said mixture (11) from the container means comprising the stirring tank (9) and static mixer (10), by means of a high pressure pump (13) that raises the pressure of the mixture from 0 kg/cm²g to a pressure of between 370.0 kg/cm²g to 380 kg/cm²g (preferably 370 kg/cm²g), wherein the conditions of the created pressurized stream (12) are as follows:

| | Microalgae Oil - |
|---|---|
| Condition | Methanol Mixture |
| Pressure, kg/cm²g | 370.00 a 380 |
| Temperature, ° C. | 58.02 (achieved by |
| | induction) |
| Mass flow, kg/h | 33.00 depending on |
| | the capacity of the |
| | pump and therefore |
| | the flow to be |
| | processed |

wherein during the start-up of the high pressure pump (13), the pressurized stream is diverted (14) through a current diverter and passes through one or more pressure regulators (15) to lower its pressure to ambient pressure and recycle it (16) to the agitation tank (9) until the pressure of 370.0 kg/cm²g is reached, at which pressure the current diverter closes the bypass (14) to the agitation tank (9) and directs it directly to a reactor (17);

d) continuously processing the homogenized mixture of methanol and microalgae oil by means of transesterification by continuously feeding the pressurized stream of the homogenized mixture of methanol and microalgae oil (12) created in step c) to a heated continuous pressurized reactor (17) whose operating conditions are: a temperature between 350° C. to 380° C. preferably 350° C. and a pressure between 370 kg/cm²g to 380 kg/cm²g (preferably 370 kg/cm²g) where the mixture is converted from methanol and microalgae oil in biodiesel with methanol and glycerin as by-products. The residence time of the continuous pressurized stream within the reactor is 120 to 200 seconds, preferably 120 seconds. At this temperature, methanol forms a homogeneous phase with the components, which eliminates diffusive problems during the reaction time. Glycerides and free fatty acids react at equivalent rates. At the outlet of the reactor, a continuous stream of a mixture of biodiesel, methanol and glycerin (18) is produced having the following conditions:

| | Biodiesel-Methanol- |
|---|---|
| Condition | Glycerin Blend |
| Pressure, kg/cm²g | 370.00 |
| Temperature, ° C. | 362.60 |
| Mass flow, kg/h | 33.00 (depending on |
| | the reactor) |

Specifically, the components of the mixture of biodiesel, methanol and glycerin are the following:

| Component | Mass fraction |
|---|---|
| Methanol | 0.9662 |
| Glycerin | 0.0032 |
| Methyl palmitate | 0.0289 |
| Methyl Oleate | 0.0008 |
| Tripalmethioyl | 0.0009 |
| Triglyceride | |
| Water | 0.0001 |
| Oleic acid | 0.0000 |
| Palmitic acid | 0.0000 |

e) depressurizing the reactor outlet stream comprising a mixture of biodiesel, methanol and glycerin (18) to a pressure of 2 kg/cm²g by passing the reactor outlet stream through one or more pressure regulation valves (19), to produce a depressurized stream (20) comprising a mixture of biodiesel, methanol and glycerin;

f) separating and purifying the biodiesel from the mixture of biodiesel, methanol and depressurized glycerin (20) obtained in step e) by means of a separation process that comprises:
a. separating the methanol from the depressurized stream (20) obtained in step e) by feeding it to a distillation column (21) where the vapor phase rich in methanol is separated at the top of the column, producing a vapor stream rich in methanol (22) and at the bottom the reaction products in liquid phase that comprise biodiesel (light liquid phase), glycerin (heavy liquid phase) and small traces of methanol (vapor phase), producing a bottom current in liquid phase (23);
b. separating the biodiesel (light liquid phase), the glycerin (heavy liquid phase) and the small traces of methanol (vapor phase) that comprise the bottom stream in the liquid phase (23), by continuously feeding said bottom stream to a three-phase separator (24) that operates at a temperature of 135° C. and a pressure of 0.50 kg/cm²g, where two output streams are produced: a first output stream that comprises biodiesel (light liquid phase) and small traces of methanol (vapor phase) (25) and a second outlet stream comprising glycerin (heavy liquid phase) and small traces of methanol (vapor phase) (26);
c. purifying the biodiesel from the first outlet stream (25) of the three-phase separator (24), separating the traces of methanol by feeding said first outlet stream to a first two-phase separator (27) that operates at a temperature of 44.47° C. where two outlet streams are produced, a first outlet stream comprising methanol in the vapor phase (28) and a second stream comprising pure biodiesel (29), which is pumped by means of a pump (30) to a tank (31) for final disposal.
The second outlet stream (26) of the three-phase separator (24) comprising glycerin (heavy liquid phase) and small traces of methanol (vapor phase) is sent to a second two-phase separator (32) where the glycerin (phase heavy liquid) and small traces of methanol (vapor phase) are separated, thus producing two outlet streams: a first outlet stream comprising methanol in vapor phase (33) and a second outlet stream comprising pure glycerin (34), which is pumped by means of a pump to a tank (35) for its final disposal.
The stream rich in methanol (22) produced at the top of the distillation column, is sent to a first plate exchanger (36) where a stream of liquid methanol (37) is produced, which is sent by means of a pump to an accumulator tank (38);
The first outlet stream (28) of the first two-phase separator (27) comprising methanol in the vapor phase, is sent to a second plate exchanger (39) by means of a pump, where a stream of liquid methanol (40) is produced, which is sent by means of a pump (41) to the accumulator tank (38);
The first outlet stream (33) of the second two-phase separator (32) comprising methanol in the vapor phase, is sent to a third plate exchanger (42) by means of a pump, where a stream of liquid methanol is produced (43), which is sent by means of a pump (41) to the accumulator tank (38);
The liquid methanol from the accumulator tank (38) is pumped to a flow separator, where a first flow of liquid methanol (44) is sent to a recovered methanol tank (45) and a reflux stream is recycled to the column distillation.

The continuous flow reactor (17) where the transesterification of the homogenized mixture of methanol and microalgae oil is carried out, has a path through which the pressurized stream flows, which is fully heated in an equitable manner by heating means.

In the specific embodiment of the process of the present invention, the continuous flow reactor comprises:

a tubular shaped outer casing (46), having: an inner space (47), an inner wall (48), a first open end (49) having a coupling ring (50) surrounding the opening; a second end (51) that ends in an open tubular extension (52) that has a smaller diameter than the outer casing (46), wherein said tubular extension (52) has an opening (not shown) surrounded by a ring of external coupling (53) and wherein the outer casing (46) has separate first (54) and second (55) upper openings arranged longitudinally in the upper portion of the outer casing (46);

a cap (56) that fits tightly to the first open end (49) of the outer casing (46) and which is attached to the coupling ring (50) of the first open end (49) by means of bolts, said cap (56) having a first (57) and a second (58) separate colonial openings disposed along a horizontal axis in an upper portion of said cap (56);

a heater element (59) comprising a tubular shaped member having a first closed end (not shown) and a second closed end (60) having a coupling disc (61), wherein the heater element (59) is housed within the interior space (47) of the outer casing (46), arranged longitudinally along the internal length of the outer casing (46) and concentrically with said outer casing (46), having a diameter such that it allows it to extend along the open tubular extension (52) in a snug manner, such that the coupling disc (61) covers the open tubular extension (52) and engages the external coupling ring (53) of said open tubular extension (52) by means of bolts. The heating element (59) includes internal heating means that in a specific embodiment comprise an electrical resistance, although in other embodiments it may comprise a gas burner;

a plurality of support rings (62) arranged equidistant, concentric and transverse within the inner shell and attached to the inner wall thereof, wherein each support ring (62) tightly holds a portion of the cross section of the heater element (59), which passes through each opening of said plurality of support rings (62);

an inner tubular core (63), composed of one or more pipes that form a circuit, said inner tubular core (63) arranged within the outer shell (46), surrounding the heating element (59) and passing through localized openings in the support rings (not shown) which serve to support the inner tubular core (63), said inner tubular core (63) having a first end (64) having a flow inlet and a second end (65) that has a processed flow outlet, where the first end (64) exits through the first opening (57) of the lid (56) in a hermetic manner and where the second end (65) exits through the second opening (58) from the lid (56) in an hermetically way;

an upper expansion tank (66), located on the outer casing (46) and connected thereto by means of a pair of valved pipes (67), (68) connected to the first and second upper openings of the outer casing.

In accordance with the specific modality of the process described above, the inlet of the inner tubular core is connected to the high pressure pump (13), and the outlet of the inner tubular core is connected to the one or more pressure regulation valves. (19).

The interior (47) of the outer casing (46) is filled with a thermal fluid, which in a preferred embodiment comprises THERMINOL-75, at a pressure of 1.5 kg/cm²g, in such a way that both the heating element and the inner tubular core are submerged in it.

In accordance with the specific embodiment of the process described above, the heating element (59) heats the thermal fluid up to a temperature of 380′C, which in turn heats the inner tubular core (63). The supercritical process by transesterification reaction is carried out continuously within the inner tubular core (63) into which the pressurized stream of the homogenized mixture of methanol and microalgae oil (12) enters through the inlet of the tubular core. internal (63). The thermal fluid heats the pressurized stream from a temperature of 58.02 to an activation temperature of 350° C. as the mixture circulates through the inner tubular core (63). Once the mixture reaches the activation temperature within the inner tubular core (63), the transesterification reaction proceeds and the conversion of reagents to Biodiesel and by-products is carried out during a residence time within the tubular core of approximately 120 seconds, which is the time it takes for a portion of the mixture to travel through the entire inner tubular core circuit (63) in such a way that as the mixture approaches the second end (65) of the inner tubular core circuit, the reaction of transesterification and therefore the conversion of reagents to Biodiesel is completely completed.

The function of the expansion tank (66) is to receive the hot oil that has expanded inside (47) of the outer casing (46) and to keep it during operation at the same reactor temperature thanks to a second element of internal heating (69) extending into the expansion tank.

It should be understood that the process of the present invention can make use of continuous flow reactors having other designs, as long as it allows the pressurized stream to reach the activation temperature and to maintain the pressure necessary to continuously carry out the transesterification reaction.

For example, the internal flow path can comprise a coil that can be heated by any suitable means other than a thermal oil.

Likewise, the stages of mixing and homogenization, depressurization, separation and purification can be carried out using any available equipment designed for this purpose and to operate continuously in conjunction with the continuous flow reactor.

It should finally be understood that the continuous process for the synthesis of biodiesel by transesterification with supercritical methanol of the present invention is not limited to the embodiment described above and that those skilled in the art will be enabled, by the teachings set forth herein, to effect changes. in the continuous process for the synthesis of biodiesel by transesterification with supercritical methanol of the present invention, the scope of which will be established exclusively by the following claims.

1. A continuous process for the synthesis of biodiesel by transesterification with supercritical methanol comprising continuously processing a pressurized stream of an homogeneous mixture of microalgae oil and methanol, by transesterification within a pressurized continuous flow reactor at a pressure of between 370 kg/cm²g to 380 kg/cm²g and at an activation temperature of between 350° C. to 380° C., in order to obtain a continuous stream of a mixture of biodiesel, methanol and glycerin.

2. (canceled)

3. (canceled)

4. (canceled)

5. A continuous process for the synthesis of biodiesel by transesterification with supercritical methanol according to claim 1 wherein once the mixture reaches the activation temperature inside the reactor, the transesterification reaction proceeds and the conversion of reagents to Biodiesel and by-products is carried out during a residence time inside the reactor of between 120 to 200 seconds, preferably 120 seconds.

6. A continuous process for the synthesis of biodiesel by transesterification with supercritical methanol according to claim 1 wherein the transesterification of the pressurized stream of a homogeneous mixture of microalgae oil and methanol, is carried out inside a continuous flow reactor comprising: a path through which the pressurized stream flows, which is fully and evenly heated by heating means.

7. A continuous process for the synthesis of biodiesel by transesterification with supercritical methanol according to claim 1, wherein the transesterification of the pressurized stream of a homogeneous mixture of microalgae oil and methanol, is carried out inside a continuous flow reactor comprising: a path through which the pressurized stream flows, which is fully and evenly heated by heating means:
and wherein once the mixture reaches the activation temperature within the path, the transesterification reaction proceeds and the conversion of reagents to biodiesel and by-products during a residence time within the path of between 120 to 200 seconds, preferably 120 seconds, which is the time it takes for a portion of the pressurized stream to travel the entire path in such a way that as the pressurized stream approaches the second end of the path, the transesterification reaction and therefore the conversion of reactants to biodiesel is fully completed.

8. (canceled)

9. (canceled)

10. (canceled)

11. A continuous process for the synthesis of biodiesel by transesterification with supercritical methanol according to claim 1 wherein the pressurized stream of a homogenized mixture of methanol and microalgae oil is generated by suction of said mixture, from a container medium by means of a high pressure pump that raises the pressure of the mixture from 0 kg/cm²g to between 370.0 kg/cm²g to 380 kg/cm²g (preferably 370 kg/cm²g), wherein the ratio of methanol to microalgae oil is from 1 to 32 with respect to the mass flow of both streams and wherein the conditions of the pressurized stream created are the following:

| | Microalgae Oil - |
|---|---|
| Condition | Methanol Mixture |
| Pressure, kg/cm²g | 370.00 a 380 |
| Temperature, ° C. | 58.02 |
| Mass flow, kg/h | 33.00 |

12. A continuous process for the synthesis of biodiesel by transesterification with supercritical methanol according to claim 1, wherein the pressurized stream of a homogenized mixture of methanol and microalgae oil is generated by suction of said mixture, from a container medium by means of a high pressure pump that raises the pressure of the mixture from 0 kg/cm²g to between 370.0 kg/cm² to 3.80 kg/cm²g (preferably 370 kg/cm²g), wherein the ratio of methanol to microalgae oil is from 1 to 32 with respect to the mass flow of both streams and wherein the conditions of the pressurized stream created are the following:

| | Microalgae Oil - |
|---|---|
| Condition | Methanol Mixture |
| Pressure, kg/cm²g | 370.00 a 380 |
| Temperature, ° C. | 58.02 |
| Mass flow, kg/h | 33.00 |

and wherein: during the start-up of the high-pressure pump, the pressurized stream is diverted by means of a current diverter and passed through a pressure regulator to lower its pressure to ambient pressure and recycle it to the container medium until the pressure of 370.0 to 380 kg/cm²g is reached, at which pressure the current diverter closes the diversion to the container medium and directs it directly to the reactor.

13. A continuous process for the synthesis of biodiesel by transesterification with supercritical methanol according to claim 1, wherein the pressurized stream of a homogenized mixture of methanol and microalgae oil is generated by suction of said mixture, from a container medium by means of a high pressure pump that raises the pressure of the mixture from 0 kg/cm²g to between 370.0 kg/cm²g to 380 kg/cm²g (preferably 370 kg/cm²g), wherein the ratio of methanol to microalgae oil is from 1 to 32 with respect to the mass flow of both streams and wherein the conditions of the pressurized stream created are the following:

| | Microalgae Oil - |
|---|---|
| Condition | Methanol Mixture |
| Pressure, kg/cm²g | 370.00 a 380 |
| Temperature, ° C. | 58.02 |
| Mass flow, kg/h | 33.00 |

and wherein
the homogenized mixture is extracted from a container means that comprises a stirring tank that has an adjoining static mixer where two streams of methanol and algae oil are mixed and homogenized that are continuously fed to said stirring tank that has a static mixer;
the methanol stream is generated by a centrifugal pump that extracts methanol from a storage tank;
the microalgae oil stream (triglyceride) is generated by a centrifugal pump that extracts microalgae oil from a storage tank;
the ratio of methanol to microalgae oil is from 1 to 32 with respect to the mass flow of both streams.

14. A continuous process for the synthesis of biodiesel by transesterification with supercritical methanol according to claim 1, comprising the subsequent step of separating and purifying the biodiesel from the mixture of biodiesel, methanol and glycerin through a separation process that comprises:
a. separating the methanol from the continuous stream of the mixture of biodiesel, methanol and glycerin that leaves the reactor but previously depressurized, wherein the methanol separation is carried out by feeding it to a distillation column wherein the vapor phase rich in methanol is separated by the head of the column, thus producing a vapor stream rich in methanol and the reaction products in the liquid phase that include biodiesel (light liquid phase), glycerin (heavy liquid phase) and small traces of methanol (vapor phase) are separated by the bottom, thus producing a bottom current in liquid phase;
b. separating biodiesel (light liquid phase), glycerin (heavy liquid phase) and the small traces of methanol (vapor phase) that comprise the bottom stream in liquid phase, by continuously feeding said bottom stream to a three-phase separator, where two outlet streams are produced: a first outlet stream comprising biodiesel (light liquid phase) and small traces of methanol (vapor phase) and a second outlet stream comprising glycerin (heavy liquid phase) and small traces of methanol (vapor phase);
c. purifying the biodiesel from the first outlet stream of the three-phase separator by separating the traces of methanol by feeding said first outlet stream to a first two-phase separator where two outlet streams are produced, a first outlet stream comprising methanol in vapor phase and a second stream comprising pure biodiesel, which is pumped through a pump to a tank for final disposal.

15. A continuous process for the synthesis of biodiesel by transesterification with supercritical methanol according to claim 1, comprising the subsequent step of separating and purifying the biodiesel from the mixture of biodiesel, methanol and glycerin through a separation process that comprises:
d. separating the methanol from the continuous stream of the mixture of biodiesel, methanol and glycerin that leaves the reactor but previously depressurized, wherein the methanol separation is carried out by feeding it to a distillation column wherein the vapor phase rich in methanol is separated by the head of the column, thus producing a vapor stream rich in methanol and the reaction products in the liquid phase that include biodiesel (light liquid phase), glycerin (heavy liquid phase) and small traces of methanol (vapor phase) are separated by the bottom, thus producing a bottom current in liquid phase;
e. separating biodiesel (light liquid phase), glycerin (heavy liquid phase) and the small traces of methanol (vapor phase) that comprise the bottom stream in liquid phase, by continuously feeding said bottom stream to a three-phase separator, where two outlet streams are produced: a first outlet stream comprising biodiesel (light liquid phase) and small traces of methanol (vapor phase) and a second outlet stream comprising glycerin (heavy liquid phase) and small traces of methanol (vapor phase);
f. purifying the biodiesel from the first outlet stream of the three-phase separator by separating the traces of methanol by feeding said first outlet stream to a first two-phase separator where two outlet streams are produced, a first outlet stream comprising methanol in vapor phase and a second stream comprising pure biodiesel, which is pumped through a pump to a tank for final disposal;
wherein the second outlet stream of the triphasic separator comprising glycerin (heavy liquid phase) and small traces of methanol (vapor phase) are feed to a second two-phase separator where glycerin (heavy liquid phase) and small traces of methanol (vapor phase) are separated, producing two outlet streams: a first outlet stream that comprises methanol in vapor phase and a second outlet stream comprising pure glycerin, which is pumped by means of a pump to a tank for its final disposal.

16. A continuous process for the synthesis of biodiesel by transesterification with supercritical methanol according to claim 441, comprising the subsequent step of separating and purifying the biodiesel from the mixture of biodiesel, methanol and glycerin through a separation process that comprises:
a. separating the methanol from the continuous stream of the mixture a biodiesel, methanol and glycerin that leaves the reactor but previously depressurized, wherein the methanol separation is carried out by feeding it to a distillation column wherein the vapor phase rich in methanol is separated by the head of the column, thus producing a vapor stream rich in methanol and the reaction products in the liquid phase that include biodiesel (light liquid phase), glycerin (heavy liquid phase) and small traces of methanol (vapor phase) are separated by the bottom, thus producing a bottom current in liquid phase;
b. separating biodiesel (light liquid phase), glycerin (heavy liquid phase and the small traces of methanol (vapor phase) that comprise the bottom stream in liquid phase, by continuously feeding said bottom stream to a three-phase separator, where two outlet streams are produced: a first outlet stream comprising biodiesel (light liquid phase) and small traces of methanol (vapor phase) and a second outlet stream comprising glycerin (heavy liquid phase) and small traces of methanol (vapor phase);
c. purifying the biodiesel from the first outlet stream of the three-phase separator by separating the traces of methanol by feeding said first outlet stream to a first two-phase separator where two outlet streams are produced, a first outlet stream comprising methanol in vapor chase and a second stream comprising pure biodiesel which is pumped through a pump to a tank for final disposal;
wherein the stream rich in methanol produced at the top of the distillation column is sent to a first plate exchanger where it is produced a stream of liquid methanol, which is sent by a pump to an accumulator tank.

17. A continuous process for the synthesis of biodiesel by transesterification with supercritical methanol according to claim 1 comprising the subsequent step of separating and purifying the biodiesel from the mixture of biodiesel methanol and glycerin through a separation process that comprises:
a. separating the methanol from the continuous stream of the mixture of biodiesel, methanol and glycerin that leaves the reactor but previously depressurized, wherein the methanol separation is carried out by feeding it to a distillation column wherein the vapor phase rich in methanol is separated by the head of the column, thus producing a vapor stream rich in methanol and the reaction products in the liquid phase that include biodiesel (light liquid phase), glycerin (heavy liquid phase) and small traces of methanol (vapor phase) are separated by the bottom, thus producing a bottom current in liquid phase;
b. separating biodiesel (light liquid phase), glycerin (heavy liquid phase) and the small traces of methanol (vapor phase) that comprise the bottom stream in liquid phase, by continuously feeding said bottom stream to a three-phase separator, where two outlet streams are produced: a first outlet stream comprising biodiesel (light liquid phase) and small traces of methanol (vapor phase) and a second outlet stream comprising glycerin (heavy liquid phase) and small traces of methanol (vapor phase);
c. purifying the biodiesel from the first outlet stream of the three-phase separator by separating the traces of methanol by feeding said first outlet stream to a first two-phase separator where two outlet streams are produced, a first outlet stream comprising methanol in vapor phase and a second stream comprising pure biodiesel, which is pumped through a pump to a tank for final disposal;
and wherein;
the first outlet stream of the first two-phase separator that comprises methanol in the vapor phase, is feed to a second plate exchanger by means of a pump, where a stream of liquid methanol is produced, which is sent by means of a pump to the accumulator tank;
the first outlet stream of the second two-phase separator comprising methanol in the vapor phase, is sent to a third plate exchanger by means of a pump, where a stream of liquid methanol is produced, which is sent by means of a pump to the accumulator tank;

18. A continuous process for the synthesis of biodiesel by transesterification with supercritical methanol according to claim 1, comprising the subsequent step of separating and purifying the biodiesel from the mixture of biodiesel methanol and glycerin through a separation process that comprises:
a. separating the methanol from the continuous stream of the mixture of biodiesel, methanol and glycerin that leaves the reactor but previously depressurized, wherein the methanol separation is carried out by feeding it to a distillation column wherein the vapor phase rich in methanol is separated by the head of the column, thus producing a vapor stream rich in methanol and the reaction products in the liquid phase that include biodiesel (light liquid phase), glycerin (heavy liquid phase) and small traces of methanol (vapor phase) are separated by the bottom, thus producing a bottom current in liquid phase;
b. separating biodiesel (light liquid phase), glycerin (heavy liquid phase) and the small traces of methanol (vapor phase) that comprise the bottom stream in liquid phase, by continuously feeding said bottom stream to a three-phase separator, where two outlet streams are produced: a first outlet stream comprising biodiesel (light liquid phase) and small traces of methanol (vapor phase) and a second outlet stream comprising glycerin (heavy liquid phase) and small traces of methanol (vapor phase);
c. purifying the biodiesel from the first outlet stream of the three-phase separator by separating the traces of methanol by feeding said first outlet stream to a first two-phase separator where two outlet streams are produced, a first outlet stream comprising methanol in vapor phase and a second stream comprising pure biodiesel, which is pumped through a pump to a tank for final disposal;
wherein the liquid methanol from the accumulator tank is pumped to a flow separator, wherein a first flow of liquid methanol is sent to a tank of recovered methanol and a reflux stream is recycled to the distillation column.

19. (canceled)

20. A continuous flow chemical reactor, comprising:
an outer shell having a tubular shape and having: an inner space, an inner wall, a first open end having a coupling ring surrounding the opening; a second end ending in a tubular extension having a smaller diameter than the outer shell, wherein said tubular extension has an opening surrounded by an external coupling ring and wherein the outer shell has separate first and second upper openings, longitudinally arranged at the upper portion of the outer shell;
a cover that is tightly fitted to the first open end of the outer shell and which is attached to the mating ring of the first open end by means of bolts, said cover having first and second separated openings arranged along ara horizontal axis at an upper portion of said cover;
a heating element comprising a tubular shaped member having a first closed end and a second closed end having a coupling disc, wherein the heating element is housed within the inner space of the outer shell, longitudinally arranged along the inner length of the outer shell and concentrically arranged with said outer shell, having a diameter such that it allows it to extend along the tubular extension in a tight manner, such that the coupling disc is located outside from the opening of the second open end of the outer casing and is coupled to the coupling ring of said open end by means of bolts;
a plurality of support rings equidistantly concentrically and transversely arranged within the inner shell and attached to the inner wall thereof, wherein each support ring tightly holds a portion of the heater element's cross-section, the which passes through the opening of the same;
an inner tubular core, composed of one or more pipes that form a circuit, said inner tubular core arranged inside the outer shell, surrounding the heating element and passing through the support rings (not its opening) which serve as support to the inner tubular core, said inner tubular core having a first end having a flow inlet and a second end having a processed flow outlet, wherein the first end exits through the first opening of the lid in a hermetic manner and wherein the second end exits through the second opening of the lid in a hermetic way; and
an upper expansion tank, located on the outer shell and connected thereto by means of a pair of pipes connected to the first and second upper openings of the outer shell;
wherein the inner tubular core is evenly heated by the heating element.

21. (canceled)

22. (canceled)

23. A continuous flow chemical reactor according to claim 20, wherein the inner space of the outer shell is filled with a thermal fluid, in such a way that both the heating element and the internal tubular core are submerged therein, which in a preferred embodiment comprises THERMINOL-75, at a pressure of 1.5 kg/cm2g, in such a way that both the heating element and the inner tubular core are submerged therein.
